(19) United States
(12) Patent Application Publication
(10) Pub. No.: US 2018/0028069 A1
(43) Pub. Date: Feb. 1, 2018

(54) Wearable thermometer patch for accurate measurement of human skin temperature

(71) Applicant: VivaLnk Inc., Santa Clara, CA (US)

(72) Inventors: Wei Shi, San Jose, CA (US); Huaquan Zhang, Hangzhou (CN); Zuofu Xu, Hangzhou (CN)

(21) Appl. No.: 15/224,121

(22) Filed: Jul. 29, 2016

(51) Int. Cl. A61B 5/01; G01K 7/30; A61B 5/00

(52) U.S. Cl. CPC A61B 5/01; A61B 5/6832; A61B 5/0008; A61B 5/0022; G01K 7/30; A61B 2562/0276

(57) ABSTRACT

A wearable thermometer patch includes a flexible circuit substrate comprising an electric circuit and an opening, a thermally conductive cup having a bottom portion plugged into the opening and fixed to the flexible circuit substrate, and a temperature sensor inside the thermally conductive cup. The temperature sensor is in thermal conduction with the thermally conductive cup. The temperature sensor is electrically connected to the electric circuit in the flexible circuit substrate.

### BACKGROUND OF THE INVENTION

The present application relates to electronic devices, and in particular, to electronic patches that can attach to human skin for conducting measurement.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime.

Electronic patches can communicate with smart phones and other devices using WiFi, Bluetooth, Near Field Communication (NFC), and other wireless technologies. NFC is a wireless communication standard that enables two devices to quickly establish communication within a short range around radio frequency of 13.56 MHz. NFC is more secure than other wireless technologies such as Bluetooth and Wi-Fi because NFC requires two devices in close proximity (e.g. less than 10 cm). NFC can also lower cost comparing to other wireless technologies by allowing one of the two devices to be passive (a passive NFC tag).

Bluetooth is another wireless communication standard for exchanging data over relatively longer distances (in tens of meters). It employs short wavelength UHF radio waves from 2.4 to 2.485 GHz from fixed or mobile devices. Bluetooth devices have evolved to meet the increasing demand for low-power solutions that is required for wearable electronics. Benefited from relatively longer reading distance and active communication, Bluetooth technologies allow wearable patches to continuously monitoring vital information without human interference, which is an advantage over NFC in many applications.

Wearable patch (or tag) is an electronic patch to be worn by a user. A wearable patch is required to stay on user's skin and operate for an extended period of time from hours to months. A wearable patch can contain a micro-electronic system that can be accessed using NFC, Bluetooth, WiFi, or other wireless technologies. A wearable patch can be integrated with different sensors such as vital signs monitoring, motion track, skin temperature measurements, and ECG detection.

Despite recent development efforts, conventional wearable patches still have several drawbacks: they may not provide adequate comfort for users to wear them; they may not stay attached to user's body for the required length of time; and they are usually not aesthetically appealing. The conventional wearable patches also include rigid polymer substrates that are not very breathable. The build-up of sweat and moisture can cause discomfort and irritation to the skin, especially after wearing it for an extended period of time.

The conventional wearable thermometer patches have the additional challenge of inaccurate temperature measurement due to factors such as thermal resistance between the temperature sensor and the human skin, conduction loss of the temperature sensor to the ambient environment, as well as temperature reduction in the user skin caused by the thermal conduction to the wearable patch. Moreover, conventional wearable thermometer patches can also have slow measurement responses.

Another challenge for conventional wearable thermometer patches is that the user's skin may interfere with their proper wireless communications. For example, the antenna's communication range can be significantly reduced by the adjacency to user's skin. The wireless communication range of an antenna in contact with the skin is less than half the range for an antenna that is placed 4 mm away from the user's skin.

There is therefore a need for a flexible wearable electronic patch that can conduct temperature measurements at user's skin with high accuracy and fast response time, while capable of performing wireless communications in a required range.

### SUMMARY OF THE INVENTION

The presently disclosure attempts to address the aforementioned limitations in conventional electronic patches. The presently disclosed wearable wireless thermometer patch that can be attached to human skin to conduct temperature measurements with high accuracy and faster respond time.

In the presently disclosed wearable wireless thermometer patch, temperature measurement errors due to the thermal noise from the environment are minimized. In metrology, accurate metrology instrument is associated with high Signal-to-Noise Ratio (SNR). In the presently disclosed wearable thermometer patch, the thermal resistance between the temperature sensor and the human skin is minimized, so that the maximum amount of heat can be conducted quickly from the user skin to the temperature sensor. Moreover, the heat conduction loss from the temperature sensor to the ambient is also minimized by the structure design and thermal material. Furthermore, a perforated protective film is placed between the user skin and the body of the wearable patch to reduce the heat conduction from the user skin, because the conventional non-perforated film will lower down the true temperature of the skin due to the attachment of the wearable patch. In addition, the presently disclosed wearable thermometer patch is structured to have low thermal capacity which results in faster responding time as well as higher flexibility.

Furthermore, the disclosed electronic patches are also breathable and stretchable. The stretchability and the breathability make the disclosed electronic patches more comfortable for the users. The disclosed electronic patches are capable wireless communication with little interference from users' skins. Moreover, the disclosed electronic patches can conduct measurements both at users' skins and away from the user's skin. The present application further discloses simple and effective manufacturing process to fabricate such wearable electronic patches.

In one general aspect, the present invention relates to a wearable thermometer patch that includes a flexible circuit substrate that has an electric circuit and an opening, a thermally conductive cup having a bottom portion plugged into the opening and fixed to the flexible circuit substrate, and a temperature sensor inside the thermally conductive cup, wherein the temperature sensor is in thermal conduction with the thermally conductive cup, wherein the temperature sensor is electrically connected to the electric circuit in the flexible circuit substrate.

Implementations of the system may include one or more of the following. The wearable thermometer patch can further include a thermally-conductive adhesive that fixes the temperature sensor to an inner surface of the thermally conductive cup; and a thermally insulating material inside a top portion of the thermally conductive cup. The temperature sensor and the thermally-conductive adhesive can be positioned near a bottom wall of thermally conductive cup, wherein the thermally insulating material can fill the top portion of the thermally conductive cup. The wearable thermometer patch can further include a flexible conductive ribbon that electrically connects the temperature sensor and to the electric circuit in the flexible circuit substrate. The wearable thermometer patch can further include a bonding pad on the flexible circuit substrate and in connection with the electric circuit, wherein the flexible conductive ribbon is connected with the bonding pad. The wearable thermometer patch can further include one or more bonding pads on an upper surface of the flexible circuit substrate, wherein the thermally conductive cup can have lips in a top portion, wherein the lips are bonded or fixedly attached to the one or more bonding pads on the upper surface of the flexible circuit substrate. The bottom portion of the thermally conductive cup can protrude out of a lower surface of the flexible circuit substrate. The thermally conductive cup can be made of a thermally conductive metallic or alloy material, a thermally conductive ceramic material, or a thermally conductive carbide composite material. The wearable thermometer patch can further include a semiconductor chip mounted on the flexible circuit substrate and in electric connection with the electric circuit, wherein the semiconductor chip can receive a first electric signal from the temperature sensor in response to measured temperature. The wearable thermometer patch can further include an antenna in electric connection with the semiconductor chip, wherein the semiconductor chip can produce a second electric signal to enable the antenna to wirelessly send the measured temperature to an external device. The antenna can be positioned near an upper surface of the flexible circuit substrate. The wearable thermometer patch can further include electronic components mounted or formed on the flexible circuit substrate and in electric connection with electric circuit, wherein the electronic components can include a semiconductor chip, an antenna, a battery, or a bonding pad; and stiffening layers formed under portions of the flexible circuit substrate which are below respective electronic components, wherein the stiffening layers can have a higher Young's modulus than the flexible circuit substrate. The wearable thermometer patch can further include an elastic layer formed on and bonded to an upper surface of the flexible circuit substrate. The wearable thermometer patch can further include electronic components mounted or formed on the flexible circuit substrate and in electric connection with electric circuit, wherein the elastic layer can include recesses on underside of the elastic layer which define cavities in which the electronic components are positioned. The flexible circuit substrate can include one or more through holes, wherein the elastic layer comprises one or more holes in registration with the one or more through holes in the flexible circuit substrate. The wearable thermometer patch can further include an adhesive layer formed on a lower surface of the flexible circuit substrate; and a layer of a perforated polymer material under the adhesive layer, wherein the layer of a perforated polymer material includes an opening to expose the a bottom portion of the thermally conductive cup. The layer of the perforated polymer material can be attached to a user′ skin, wherein the layer of the perforated polymer material, the flexible circuit substrate, and the elastic layer can include one or more holes configured to bring moisture from the user's skin to environment above the elastic layer.

These and other aspects, their implementations and other features are described in detail in the drawings, the description and the claims.

### BRIEF DESCRIPTION OF THE DRAWINGS

FIG. 1 illustrates the usage of a wearable patch attached to a user's skin.

FIG. 2 is a cross-sectional view of a base structure for constructing a wearable thermometer patch in accordance with some embodiments of the present invention.

FIG. 3 is a cross-sectional view of a wearable thermometer patch capable of conducting accurate and fast-response temperature measurements and effective wireless communications in accordance with some embodiments of the present invention.

FIG. 4 is a detailed cross-sectional view of the temperature sensing portion in the wearable thermometer patch in FIG. 3.

### DETAILED DESCRIPTION OF THE INVENTION

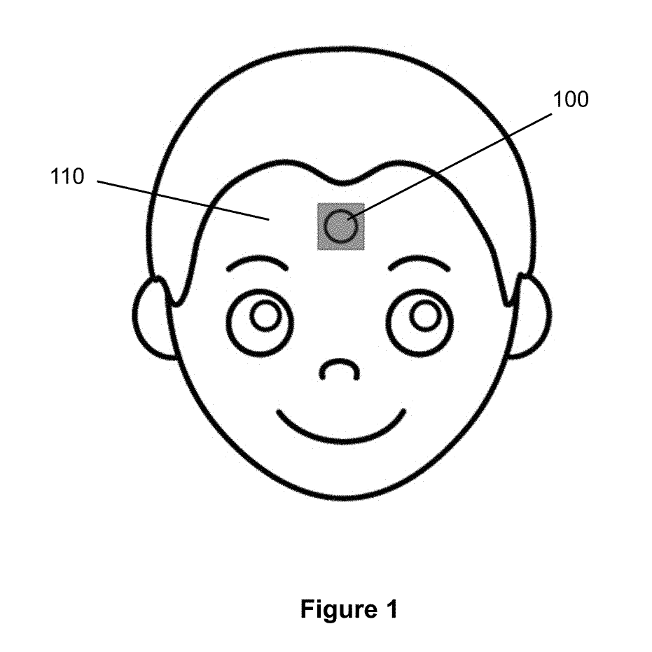

Referring to FIG. 1, a wearable patch 100 is attached to a user's skin 110 for measuring body vital signs. The wearable patch 100 can be placed on forehead, hand, wrist, arm, shoulder, waist, leg, foot, or other parts of the body. In the present disclosure, the term “wearable patch” can also be referred to as “wearable sticker” or “wearable tag”.

As discussed above, wearable electronic patches face several challenges: the user's skin 110 may interfere with their proper operations. For example, the wearable patch 100 may include an antenna for wireless communications with other devices. The antenna's communication range can be significantly reduced when an antenna is placed in contact with the user's skin 110.

The presently disclosure aims to overcome the drawbacks in conventional wearable patches, and to provide highly stretchable, compliant, durable, breathable, and comfortable wearable electronic patches while performing more accurate and more responsive measurements and communication functions.

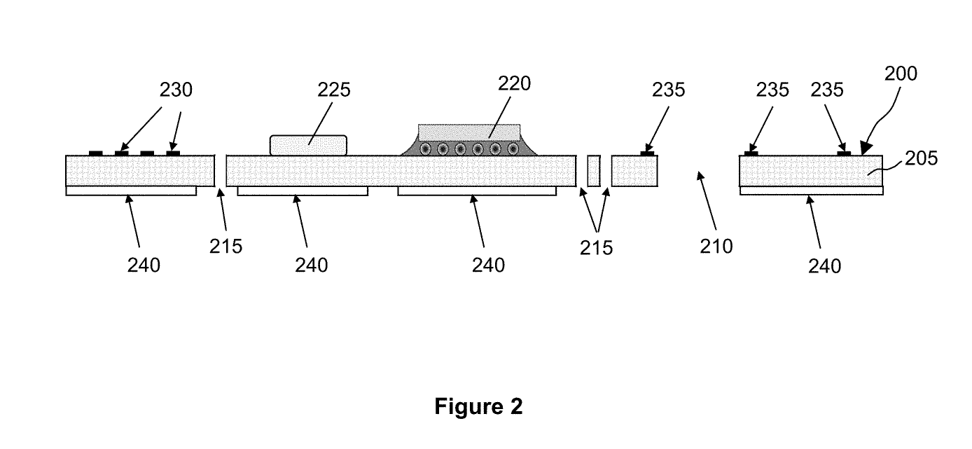

Referring to FIG. 2, a base structure 200 includes a flexible circuit substrate 205 having an electric circuit embedded in or formed on. The flexible circuit substrate 205 has a large opening 210 and multiple small through holes 215. A semiconductor chip 220, a battery 225, an antenna 230, and bonding pads 235 are mounted or formed on the upper surface of the flexible circuit substrate 205. The semiconductor chip 220, the battery 225, the antenna 230, and at least one of the bonding pads 235 is connected with the electric circuit in the flexible circuit substrate 205.

Stiffening layers 240 are formed on the layer surface of the flexible circuit substrate 205 at locations respectively below electronic components such as the semiconductor chip 220, the battery 225, the antenna 230, and the bonding pads 235. The stiffening layers 240 have higher Young's modulus than that of the flexible circuit substrate 205, and can protect the electronic devices from being damaged when the flexible circuit substrate 205 is bent. The flexible circuit substrate 205 can be made of polymeric materials and built in with electric circuitry that connects the semiconductor chip 220, the battery 225, the antenna 230, and the bonding pads 235. The stiffening layers 240 can be made of metallic or polymeric materials.

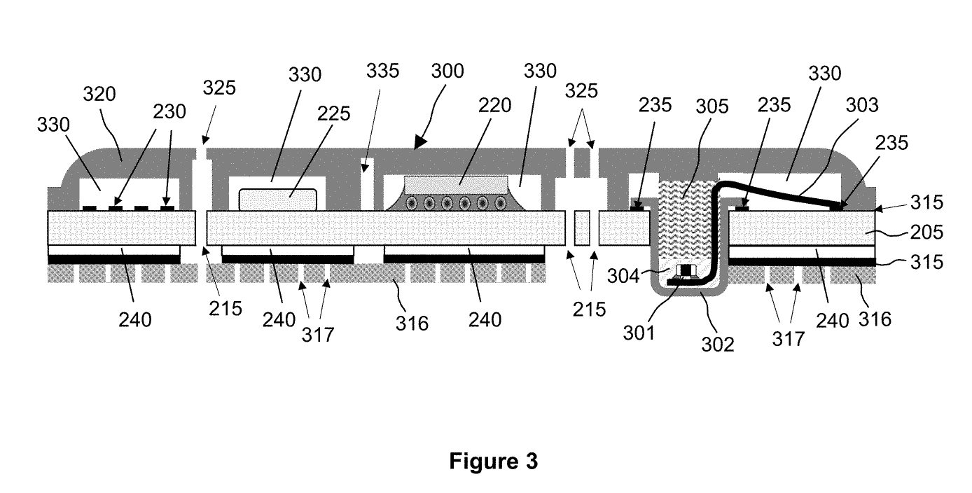

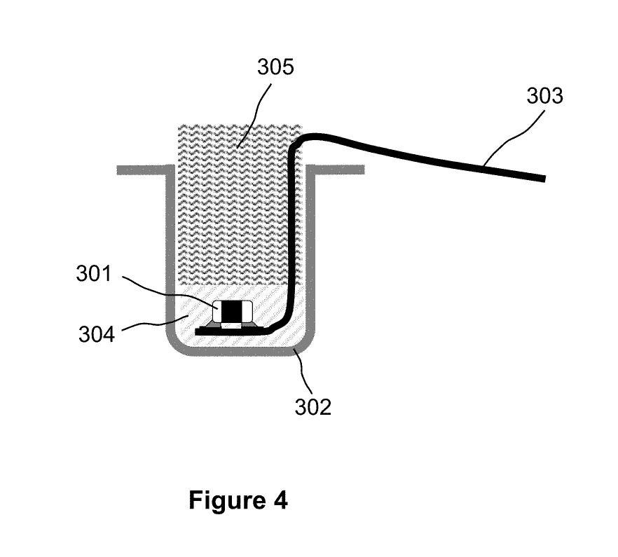

Referring to FIGS. 3 and 4, a wearable thermometer patch 300 that includes an assembly for temperature sensing, in addition to the components in the base structure 200 as shown in FIG. 2. A thermally conductive cup 302 has its bottom portion plugged into the large opening 210 (FIG. 2). The bottom portion of the thermally conductive cup 302 protrudes out of the lower surface of the flexible circuit substrate 205. The lips of the thermally conductive cup 302 near its top portion are fixedly attached or bonded to bonding pads 235 by soldering or with an adhesive. The thermally conductive cup 302 can be made of a thermally conductive metallic or alloy material such as copper, stainless steel, ceramic or carbide composite materials. A temperature sensor 301 is attached to and in thermal conduction with an inner surface near the bottom of the thermally conductive cup 302. The temperature sensor 301 can be implemented, for example, by a Thermistor, a Resistor Temperature Detector, or a Thermocouple. When an outer surface of the bottom portion of the thermally conductive cup 302 is in contact with a user's skin, the thermally conductive cup 302 can thus effectively transfer heat from a user's skin to the temperature sensor 301. A flexible conductive ribbon 303 is connected to the temperature sensor 301 in the thermally conductive cup 302 and one of the conductive pads 235 on the flexible circuit substrate 205. Thus the temperature sensor 301 is connected to the electric circuit in the flexible circuit substrate 205 and can send an electric signal to the electric circuit and the semiconductor chip 220 in response to temperature measured by the temperature sensor 301. The semiconductor chip 220 processes the electric signal and output another electrical signal which enables the antenna 230 to transmit a wireless signal to send measurement data to another external device such as a mobile phone or a computer. The battery 225 powers the semiconductor chip 220, the electric circuit, and possibly the temperature sensor 301.

The temperature sensor 301 and a portion of the flexible conductive ribbon 303 are fixed to an inner surface at the bottom of the thermally conductive cup 302 by a thermally-conductive adhesive 304, which allows effective heat transfer from the bottom of the thermally conductive cup 302 to the temperature sensor 301. Examples of the thermally-conductive adhesive 304 can include electrically-insulative thermally-conductive epoxies and polymers. A thermally insulating material 305 is fixed in and fills the top portion of the thermally conductive cup 302, which fixes the thermally-conductive adhesive 304 at the bottom of the thermally conductive cup 302 and reduces heat loss from the temperature sensor 301 to the elastic layer (described below) or the environment. The flexible conductive ribbon 303 can be bent and laid out along the wall the thermally conductive cup 302.

A layer of a perforated polymer material 316 is bonded to the bottom surface of the flexible circuit substrate 205 using adhesive material 315. Suitable material for the perforated polymer material 316 can include soft materials such as Polyurethane. The layer of perforated polymer material 316 can include multiple holes 317: one of them exposes a bottom of the thermally conductive cup; others allow sweat and moisture to escape through holes 215 and holes 325; while other holes 317 help enhance flexibility and comfort of the perforated polymer material. An adhesive material is applied to the lower surface of the perforated polymer material 316 to be attached the lower surface of the perforated polymer material 316 to the user's skin, so that the bottom of the thermally conductive cup 302 can be in tight contact with a user's skin for the accurate temperature measurement of the user's skin.

It should be noted that when the wearable thermometer patch 300 is worn by the user, the antenna 230 is separated from the user's skin by the flexible circuit substrate 205 and the layer of the perforated polymer material 316, which minimizes the impact of the user's body on the transmissions of wireless signals by the antenna 230.

An elastic layer 320 is bonded onto the upper surface of the flexible circuit substrate 205 with an adhesive material 315 in between. Alternatively, the elastic layer 320 can directly be molded onto the flexible circuit substrate 205 without using any bonding interface material 315. The elastic layer 320 includes recesses 330 on the underside to define cavities to contain the antenna 230, the battery 225, the semiconductor chip 220 and the flexible conductive ribbon 303. The elastic layer 320 also includes holes 325 that are registered to the through holes 215 in the flexible circuit substrate 205, which allows moisture and sweat from the user's skin to diffuse to the ambient environment, which enhances user's comfort and strength of attachment of the wearable thermometer patch 300 to the user's skin. The elastic layer 320 can include one or more cavities 335 for enhancing flexibility (bendable) and stretchability of the elastic layer 320 and the whole wearable thermometer patch 300. The cavities 335 can have elongated shapes with lengthwise direction oriented perpendicular to the flexible circuit substrate 205.

The elastic layer 320 can be made of a non-conductive material such as an elastomeric material or a viscoelastic polymeric material having low Young's modulus and high failure strain. In some embodiments, the elastic layer 320 has a Young's Modulus <0.3 Gpa. In some cases, the elastic layer 320 and can have Young's Modulus <0.1 Gpa to provide enhanced flexibility and tackability. Materials suitable for the elastic layer 320 include elastomers, viscoelastic polymers, such as silicone, silicone rubber, and medical grade polyurethane that is a transparent medical dressing used to cover and protect wounds with breathability and conformation to skin.

The disclosed wearable thermometer patch can significantly enhance measurement accuracy and responsiveness, and reduce thermal noise. The temperature sensor is positioned very close to a user's skin. The temperature sensor is placed at the bottom of a thermally conductive cup and in good thermal conduction with the user's skin. The minimized thermal resistance between the temperature sensor and the user's skin reduces temperature measurement error and also decreases measurement response time. Moreover, the temperature sensor is secured fixed by an adhesive to the bottom of the thermally conductive cup such that the temperature sensor is not affected and detached by user's body movements, which improves durability of the wearable thermometer patch. Furthermore, the temperature sensor is thermally isolated with the ambient environment by a thermal insulating material in the top portion of the thermally conductive cup. The reduced thermal capacity helps further reduces background noise in the measurements of user's skin temperature and increase response rate of measurement. A layer of soft perforated polymer material under the flexible substrate minimizes heat conduction from the user's skin to the wearable thermometer patch, thus reducing the “cooling effect” of the user's skin by the wearable thermometer patch.

Another advantage of the disclosed wearable thermometer patch is that it is stretchable, compliant, durable, and comfortable to wear by users. The disclosed wearable thermometer patch includes a flexible substrate covered and protected by an elastic layer that increases the flexibility and stretchability. Cavities within the elastic layer further increase its flexibility and stretchability. A layer of soft perforated polymer material under the flexible substrate provides comfortable contact to user's skin is in contact with user's skin. Openings in the elastic layer, the substrate, and the soft perforated polymer material can bring moisture and sweat from the user's skin to the ambient environment, which increases user's comfort as well as strength of the attachment of the wearable thermometer patch to user's skin.

Yet another advantage of the disclosed wearable thermometer patch is that it can significantly increase wireless communication range by placing the antenna on the upper surface of the flexible circuit substrate. The thickness of the substrate as well as the height of the thermally conductive cup can be selected to allow enough distance between the antenna and the user's skin to minimize interference of user's body to the wireless transmission signals.

Further details of wearable thermometer patches are disclosed in the commonly assigned co-pending U.S. patent application Ser. No. 14/814,347 “Three dimensional electronic patch”, filed Jul. 30, 2015, the disclosure of which is incorporated herein by reference.

The disclosed wearable thermometer patches can also include electronic components such as the semiconductor chips, resistors, capacitors, inductors, diodes (including for example photo sensitive and light emitting types), other types of sensors, transistors, amplifiers. The sensors can also measure temperature, acceleration and movements, and chemical or biological substances. The electronic components can also include electromechanical actuators, chemical injectors, etc. The semiconductor chips can perform communications, logic, signal or data processing, control, calibration, status report, diagnostics, and other functions.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A wearable thermometer patch, comprising:
a flexible circuit substrate comprising an electric circuit and an opening;
a thermally conductive cup having a bottom portion plugged into the opening and fixed to the flexible circuit substrate; and
a temperature sensor inside the thermally conductive cup, wherein the temperature sensor is in thermal conduction with the thermally conductive cup, wherein the temperature sensor is electrically connected to the electric circuit in the flexible circuit substrate.

2. The wearable thermometer patch of claim 1, further comprising:
a thermally-conductive adhesive that fixes the temperature sensor to an inner surface of the thermally conductive cup; and
a thermally insulating material in a top portion of the thermally conductive cup.

3. The wearable thermometer patch of claim 2, wherein the temperature sensor and the thermally-conductive adhesive are positioned near a bottom wall of thermally conductive cup, wherein the thermally insulating material fills the top portion of the thermally conductive cup.

4. The wearable thermometer patch of claim 1, further comprising:
a flexible conductive ribbon that electrically connects the temperature sensor to the electric circuit in the flexible circuit substrate.

5. The wearable thermometer patch of claim 4, further comprising:
a bonding pad on the flexible circuit substrate and in connection with the electric circuit, wherein the flexible conductive ribbon is connected with the bonding pad.

6. The wearable thermometer patch of claim 1, further comprising:
one or more bonding pads on an upper surface of the flexible circuit substrate,
wherein the thermally conductive cup has lips in a top portion, wherein the lips are bonded or fixedly attached to the one or more bonding pads on the upper surface of the flexible circuit substrate.

7. The wearable thermometer patch of claim 1, wherein the bottom portion of the thermally conductive cup protrudes out of a lower surface of the flexible circuit substrate.

8. The wearable thermometer patch of claim 1, wherein the thermally conductive cup is made of a thermally conductive metallic or alloy material, a thermally conductive ceramic material, or a thermally conductive carbide composite material.

9. The wearable thermometer patch of claim 1, further comprising:
a semiconductor chip mounted on the flexible circuit substrate and in electric connection with the electric circuit, wherein the semiconductor chip is configured to receive a first electric signal from the temperature sensor in response to measured temperature.

10. The wearable thermometer patch of claim 9, further comprising:
an antenna in electric connection with the semiconductor chip, wherein the semiconductor chip is configured to produce a second electric signal to enable the antenna to wirelessly send the measured temperature to an external device.

11. The wearable thermometer patch of claim 10, wherein the antenna is positioned near an upper surface of the flexible circuit substrate.

12. The wearable thermometer patch of claim 1, further comprising:
electronic components mounted or formed on the flexible circuit substrate and in electric connection with electric circuit, wherein the electronic components comprise a semiconductor chip, an antenna, a battery, or a bonding pad; and
stiffening layers formed under portions of the flexible circuit substrate which are below respective electronic components, wherein the stiffening layers have a higher Young's modulus than the flexible circuit substrate.

13. The wearable thermometer patch of claim 1, further comprising:
an elastic layer formed on and bonded to an upper surface of the flexible circuit substrate.

14. The wearable thermometer patch of claim 13, further comprising:
electronic components mounted or formed on the flexible circuit substrate and in electric connection with electric circuit,
wherein the elastic layer comprises recesses on underside of the elastic layer which define cavities in which the electronic components are positioned.

15. The wearable thermometer patch of claim 14, wherein the flexible circuit substrate comprise one or more through holes, wherein the elastic layer comprises one or more holes in registration with the one or more through holes in the flexible circuit substrate.

16. The wearable thermometer patch of claim 1, further comprising:
an adhesive layer formed on a lower surface of the flexible circuit substrate; and
a layer of a perforated polymer material under the adhesive layer, wherein the layer of a perforated polymer material includes an opening to expose the bottom portion of the thermally conductive cup.

17. The wearable thermometer patch of claim 16, wherein the layer of the perforated polymer material is configured to be attached to a user′ skin, wherein the layer of the perforated polymer material, the flexible circuit substrate, and the elastic layer comprise one or more holes configured to bring moisture from the user's skin to environment above the elastic layer.
